(19) United States
(12) Patent Application Publication
(10) Pub. No.: US 2010/0310479 A1
(43) Pub. Date: Dec. 9, 2010

(54) USE OF CERTAIN ISOPROPYL METHYLCYCLOHEXENE THIOLS AS FRAGRANCES AND/OR FLAVORS

(75) Inventors: Frank Ott, Holzminden (DE); Günter Kindel, Hoxter (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/793,282

(22) Filed: Jun. 3, 2010

(30) Foreign Application Priority Data
Jun. 3, 2009 (DE) 102009026698.4

(51) Int. Cl. A61K 8/46; C07C 321/22; A61Q 90/00; A61Q 17/04; A61Q 5/00; A61Q 15/00; A61Q 1/06; A61Q 1/10; A61Q 19/04; A61Q 9/02; A23L 1/226; A61Q 13/00

(52) U.S. Cl. 424/49; 568/61; 514/706; 424/59; 424/70.1; 424/65; 424/64; 424/70.7; 424/73; 512/7; 426/650

(57) ABSTRACT

A description is provided of new compounds of formula (A) and in particular the use of a compound of formula (A) or a mixture of two or more compounds of formula (A), wherein the thiol group of each compound of formula (A) independently of one another in each case is linked with the carbon atom in position 3, 4, 5 or 6, as a fragrance or flavor for imparting, modifying and/or intensifying one, two or all the smell and/or taste notes grapefruit, tropical fruits and blackcurrant

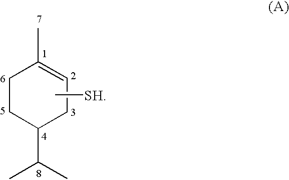

Also described are fragrance and flavor compositions, preparations and methods.

The present invention primarily relates to the use of certain isopropyl methylcyclohexene thiols, namely compounds of formula (A) as illustrated below, as fragrances and/or flavors, in particular for imparting, modifying and/or intensifying a flowery and/or fruity taste and/or smell note, preferably one, two or all the smell and/or taste notes grapefruit, tropical fruits and blackcurrant.

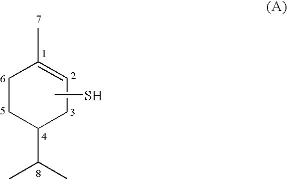

Compounds of formula A to be used by preference are described further in the following.

Additionally the invention relates to the new compounds of formulas (III) and (IV) (as described in each case below) and mixtures comprising or consisting of one or more compounds of formula (III) or (IV) and as necessary one or more compounds of formula (I) or (II).

A further aspect of the present invention relates to fragrance or flavor compositions, comprising one or more inventive or inventively used compounds of formula (A). Furthermore, the present invention relates to preparations and fragranced items for nutrition, pleasure or oral hygiene, comprising one or more compounds of formula (A) or an inventive fragrance or flavor composition.

In addition the present invention relates to a method for imparting, modifying and/or intensifying a smell and/or taste and a method for producing compounds of formula (A).

Further aspects of the present invention and particularly preferred embodiments will arise out of the following description, the examples and the attached claims.

Despite the existence of a number of fragrances and flavors in both the perfume and food-processing industries there continues to be a general demand for new fragrances and flavors which in addition to their primary, that is to say olfactory or taste properties, also have positive secondary properties such as, for example, increased stability and under certain usage conditions, a higher substantialness and a better adhesive power, but also improved dermatological and toxicological properties with respect to comparable fragrances or flavors.

There is a particular demand for fragrances and flavors which have the olfactory or taste characteristics of citrus fruits.

The taste characteristics of citrus fruits (Citrus aurantium), in particular lemon, orange, Citrus sinensis, mandarin, clementine, grapefruit, Citrus maxima, lime, sweet lime, kumquat, tangor and tangelo, are characterized by a series of mono-, sesqui-, di- and triterpenes. A large number of these compounds can be found in the commercially available oils and extracts of the various fruits.

One of the most prominent compounds among the sulfurous terpenes is 2-(4-methyl-cyclohex-3-enyl)-propane-2-thiol, better known in terpene chemistry by the usual name p-menth-1-ene-8-thiol or also p-menthene thiol-1,8 (compound G, see below) (see Helv. Chim. Acta 1982, 65(6), 1785-1794). p-menthene thiol-1,8 is sensorially active in the low ppb-range and is an important compound for a typical, fresh grapefruit note. It is known, however, that p-menthene thiol-1,8 in acid medium, as is also the case in natural fruit, reacts easily with 1,8-epithio-p-menthane and/or with 2,8-epithio-p-menthane (see Helv. Chim. Acta 1982, 65(6), 1785-1794), as shown in the following diagram:

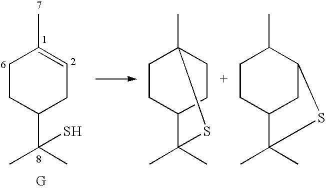

The episulfides formed here have sensory properties that in relation to compound G have (often) undesirable notes such as leek, onion and durian. As a result the sensory profile of p-menthene thiol-1,8 (compound G), that is to say the fresh grapefruit note, is quickly lost.

The object of the present invention was therefore to find new fragrances or flavors, in particular fragrances or flavors having similar sensory properties to p-menthene thiol-1,8. Accordingly fragrances or flavors were preferably to be found that were able to impart, modify and/or intensify a fruity smell and/or taste note, in particular a grapefruit note. More preferably here flavors and fragrances were to be found which had a higher stability than p-menthene thiol-1,8 (in acid medium). Further objects of the invention will arise out of the following description, the examples and in particular the attached claims.

The primary object of the present invention is achieved according to the invention by the use of
a compound of formula (A)

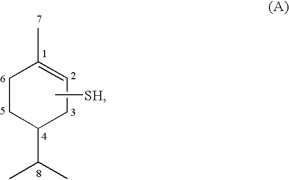

wherein the thiol group of the compound of formula (A) is linked with the carbon atom in position 3, 4, 5 or 6 (according to the numbering shown),
   or
a mixture of two or more compounds of formula (A), wherein the thiol group of each compound of formula (A) independently of one another in each case is linked with the carbon atom in position 3, 4, 5 or 6 (according to the numbering shown),
   as a fragrance or flavor for imparting, modifying and/or intensifying a flowery and/or fruity smell and/or taste notes, preferably for imparting, modifying and or intensifying one, two or all the smell and/or taste notes grapefruit, tropical fruits and blackcurrant.

In the illustration provided above of formula (A) the numbers 1 to 8 correspond to an arbitrary, not necessarily IUPAC-conforming, nomenclature of the carbon atoms.

With the compounds of formula (A) (as shown above) it is a case, according to the position of the thiol group, of the compounds of formulas (I) to (IV):

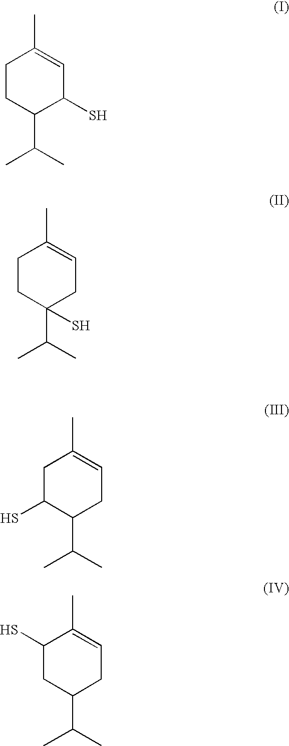

Accordingly, the compounds of formula (A) to be used inventively comprise the compounds
4-isopropyl-1-methyl-cyclohex-1-ene-3-thiol (compound of formula (I), also p-menth-1-ene-3-thiol or p-menthene thiol-1,3),
4-isopropyl-1-methyl-cyclohex-1-ene-4-thiol (compound of formula (II), also p-menth-1-ene-4-thiol or p-menthene thiol-1,4),
4-isopropyl-1-methyl-cyclohex-1-ene-5-thiol (compound of formula (III), also p-menth-1-ene-5-thiol or p-menthene thiol-1,5) and
4-isopropyl-1-methyl-cyclohex-1-ene-6-thiol (compound of formula (IV), also p-menth-1-ene-6-thiol or p-menthene thiol-1,6) and enantiomers and diastereomers of these.

In the Flavour and Fragrance Journal 1993, 8, 289-294 the conversion of a number of terpenes with hydrogen sulfide and catalysts such as aluminum trichloride was investigated. In the process sulfurous terpenes were produced and the raw product was analyzed under mass spectrometry, in the process of which the compounds of formula (I) (as shown above) were also found in varying proportions by volume in the product mixtures produced there. Depending on the terpenes selected for use, reaction mixtures were obtained there which apart from one or more compounds of formula (I) also contained one or more fragrances or flavors such as limonene, alpha-pinene, alpha-terpinene, gamma-terpinene, 3-carene, p-cymene, 1,8-cineol (eucalyptol) and p-menthene thiol-1,8 (compound G, see above). Furthermore, in the cited publication a compound of formula (II) (as shown above) and reaction mixtures containing such a compound are described. In this publication the authors expressly refer to the fact that the investigation of the sensory properties of the compounds obtained were not the focus of their work, so that in this citation no description of the sensory properties of compounds of formula (I) or (II) is found either.

The reaction mixtures manufactured or manufacturable by conversion starting respectively with limonene, alpha-pinene, alpha-terpinene, gamma-terpinene or 3-carene with hydrogen sulfide in the presence of aluminum trichloride as per the Flavour and Fragrance Journal 1993, 8, 289-294 and the other mixtures disclosed in that document (e.g. the mixtures used for liquid chromatography with hexane or ethyl acetate) are expressly excluded from the protection of the present invention. Furthermore these mixtures are not (inventive) preparations serving for nutrition, pleasure or oral hygiene or perfumed items (as described further below).

The following table shows the sensory properties of the inventive compounds of formulas (I) to (IV) determined in our own investigations. For internal tastings to determine the sensory properties the compounds to be tasted of formulas (I) to (IV) were used in each case in a quantity of 500 ppb in 5% sugar solution and 0.5% saline solution. The strength of the respective (sensory) impressions is indicated on a scale of 1 (very weak) to 9 (very strong).

| Compound | Sensory description |
|---|---|
| Formula (I) | impact (7), intensity (6), fullness (6), adherent (5), grapefruit (5), bucco |
| | (5), sulfurous (5), overripe (5), testaceous (5), tropical fruits (4), passion |
| | fruit (4), roasted (4), fruity (3), blackcurrant (3), citrus (3), sulfurous (3) |
| Formula (II) | intensity (6), impact (6), adherent (6), fullness (5), grapefruit (5), |
| | testaceous (5), sulfurous (4), bucco (3), tropical fruits (3), blackcurrant (3), |
| | citrus, sweet lime (2) |
| Formula (III) | adherent (7), intensity (8), impact (7), fullness (6), citrus (6), grapefruit |
| | (6), bucco (6), tropical fruits (5), herbaceous (5), fruity (5), passion fruit |
| | (4), blackcurrant (4), sweet (3), sulfurous (3) |
| Formula (IV) | intensity (8), impact (8), grapefruit (8), sulfurous (8), adherent (7), |
| | fullness (7), blackcurrant (7), tropical fruits (7), bucco (6), guava (6), |
| | cassis (6), sesame, roasted (4), citrus (2) |

In comparison the sensory properties of the compound G (p-menthene thiol-1,8, see above) known from the state of the art according to our investigations can be described as follows:

grapefruit (8), intensity (7), impact (6), adherent (5), fullness (5), fruity (5), sweet lime (4), tropical fruits (4), juicy (3), citrus (3), mango (3), blackcurrant (3), bucco (3), passion fruit (3), sweet (3), sulfurous (3), durian (2).

In addition, in our own investigations we investigated the sensory properties of the known compound L from the state of the art (see below)

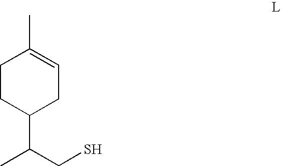

In the process of these the compound L exhibited strong fruity (in the direction of apple and tropical fruits), sulfurous and herbaceous notes, but also fresh-green and burnt-woody aspects. The sensory profile of compound L thus differs in a surprisingly distinct way from the sensory profile of the compounds of formula (A) to be used inventively.

Particularly surprisingly, during our own investigations, it was found that with the compounds of formula (A) to be used inventively—in comparison with p-menthene thiol-1,8 (compound G, see above)—the grapefruit note (in particular in acid medium) remained stable. On the basis of Helv. Chim. Acta 1982, 65(6), 1785-1794 (see above), it could not be expected that through a different arrangement of the thiol groups compared with p-menthene thiol-1,8, more stable fragrances or flavors will be obtained.

In addition, during our own investigations we found that depending on the position of the thiol group, that is to say depending on whether the thiol group is linked with the carbon atom in position 3, 4, 5 or 6 (as described above), a(n at least partially) different sensory profile results or individual sensory function descriptors can be expressed in a more intense, modified or weaker manner. The complexity of the sensory profiles of the inventive or inventively used compounds of formula (A) advantageously allows, through the selection of a compounds of formula (A) particularly suited to the desired application or of a mixture of two or more compounds of formula (A) (as described in each case above), a particular sensory function to be advantageously emphasized.

An inventively used compound of formula (I) (as described above), apart from typical grapefruit notes (similar to those of p-menthene thiol-1,8), also advantageously has sulfurously testaceous nuances and bucco and blackcurrant notes, but also tropical fruit notes.

The inventively used compound of formula (II) (as described above), thanks to its citric grapefruit and sweet lime character, combined with leek and onion-like sulfurous notes, advantageously also has particularly interesting aspects.

An inventive or inventively used compound of formula (III) has a similar sensory characteristic to a compound of formula (I), but with regard to the citrus, tropical fruits and bucco aspects, is considerably more dominant. Similarly, through a compound of formula (III) advantageously sulfurously dusty notes of fresh grapefruits or grapefruit peel can be brought out and sustained with particular persistence.

In particular, an inventive or inventively used compound of formula (IV) advantageously imparts a markedly intensive impression of blackcurrants.

The inventive or inventively used compounds of formulas (III) and (IV) are new, inventive compounds compared with the state of the art (on this point see below).

According to the invention preference is for a use (as described above), wherein the compound of formula (A) or one, a plurality of or all compounds of formula (A) in the mixture is selected or are selected independently of one another in each case from the group consisting of compounds of formulas (I) and (III)

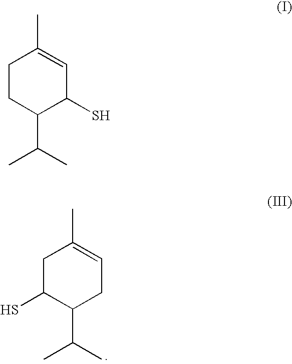

Particular preference is for an inventive use (as described above), wherein the compound of formula (A) or one, a plurality of or all compounds of formula (A) in the mixture is selected or are selected independently of one another in each case from the group consisting of the compounds of formulas (Ia), (Ib), (Ic), (Id), (IIIa), (IIIb), (IIIc) and (IIId)

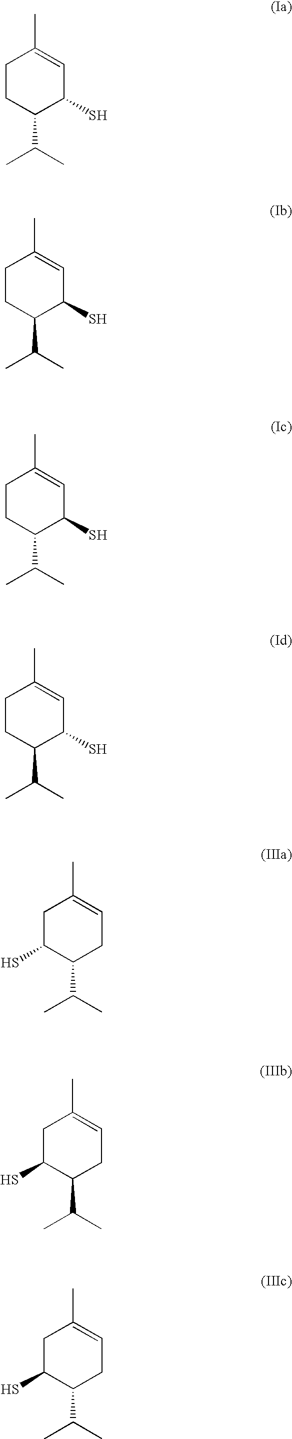

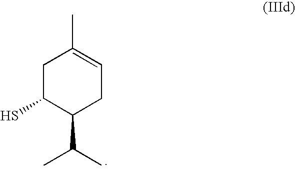

According to the invention, particularly preferred compounds of formula (A) are accordingly compounds of formulas (I) and (III), in particular their respective stereoisomers (Ia) to (Id) and (IIIa) to (IIId).

Particular preference is also for an inventive use of a mixture of two or more compounds of formula (A) (as described above), wherein the mixture comprises or consists of

one or more compounds of formula (A), selected from the group consisting of the compounds of formulas (Ia), (Ib), (Ic) and (Id), and/or
one or more compounds of formula (A), selected from the group consisting of the compounds of formulas (IIIa), (IIIb), (IIIc) and (IIId),
preferably selected from the group consisting of the compounds of formulas (Ib), (Id), (IIIb) and (IIId).

Such mixtures to be preferably used according to the invention have a particularly advantageous and sought-after sensory profile.

The stated advantages apply in particular for a further inventively preferred use (as described above), wherein the mixture comprises or consists of one or more compounds of formula (A), selected from the group consisting of the compounds of formulas (Ia), (Ib), (Ic) and (Id), and one or more compounds of formula (A), selected from the group consisting of the compounds of formulas (IIIa), (IIIb), (IIIc) and (IIId) and

wherein the ratio by weight of the total quantity of compounds of formula (III) to the total quantity of compounds of formula (I) in the mixture is preferably in the range 1:99 to 20:80, preferably in the range 2:98 to 10:90, and particularly preferably in the range 3:97 to 8:92.

A further aspect of the present invention relates to a method for producing inventive or inventively used compounds of formula (A), comprising the following steps:

(1) conversion of a compound of formula (B) or (C)

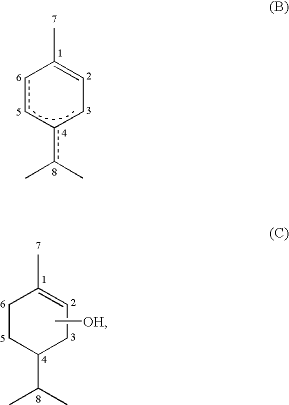

wherein in formula (B) the broken lines indicates a (second) double bond between two neighboring carbon atoms within the cyclohexane ring, that is to say between the carbon atoms in positions 6 and 5, 5 and 4 or 4 and 3, or outside of the cyclohexane ring, that is to say between the carbon atoms in positions 4 and 8,
wherein in formula (C) (piperitol and hydroxy-position isomers) the OH group is bonded to a carbon atom in one of positions 3, 4, 5 or 6,
with thioacetic acid (AcSH), if necessary in the presence of a zinc compound,
and
(2) alkaline hydrolysis of the thioacetates obtained in step (1) with a base, preferably selected from the group consisting of LiOH, NaOH and Ca(OH)₂, and a solvent, preferably comprising or consisting of water and an organic solvent that is (at least partially) miscible with water, preferably selected from the group consisting of THF, dioxane, acetone and methanol.

Where step (1) is carried out in the presence of a(n anhydrous) zinc compound, it is preferably a case here of a zinc halogenide, more preferably zinc iodide (ZnI₂), zinc bromide (ZnBr₂) or zinc chloride (ZnCl₂).

As is known from the state of the art (see, for example, Tetrahedron: Asymmetry 2001, 12, 677-683 and Tetrahedron: Asymmetry 2000, 11, 3591-3607) compounds of formula (C) can also be produced that are enantiopure. When an enantiopure compound of formula (C) is used in step (1), the inventively used compounds of formula (A), preferably the compounds of formulas (I) and (III), can be produced in an enantiopure or at least enantioenriched manner.

Thus a compound of formula (I) (as described above) to be preferably used according to the invention, can be produced for example starting with piperitone or piperitol in an enantiopure or enantioenriched manner (see Tetrahedron: Asymmetry 2001, 12, 677-683).

A mixture to be preferably used according to the invention comprising one or more compounds of formula (I) and one or more compounds of formula (III) (as described above), starting with piperitol, which can be obtained by example by the reduction of piperitone (Tetrahedron: Asymmetry 2001, 12, 677-683), can be produced in an enantiopure or enantioenriched manner.

In connection with the present invention, in our own investigations, for example starting with enantiopure piperitol, through the conversion with thioacetic acid in the presence of anhydrous zinc iodide and subsequent alkaline hydrolysis of the thioacetate formed during the conversion (following chromatographic purification), a mixture is produced comprising 57.5% by weight of compound of formula (Id), 33.9% by weight of compound of formula (Ib), 3.2% by weight of compound of formula (IIId) and 2.3% by weight of compound of formula (IIIb).

In a corresponding manner, starting with racemic piperitone over piperitol following chromatographic purification a mixture comprising 28.9% by weight compound of formula (Id), 28.9% by weight compound of formula (Ic), 17.1% by weight compound of formula (Ib), 17.1% by weight compound of formula (Ia), 1.6% by weight compound of formula (IIId), 1.6% by weight compound of formula (IIIc), 1.2% by weight compound of formula (IIIb) and 1.2% by weight compound (of formula (IIIa)) is produced.

The inventively used compounds of formula (A) are advantageously particularly well suited to use in preparations for nutrition or pleasure and as a component of a fragrance or flavor composition.

Accordingly, the present invention relates in particular to an inventive use (as described above) in a fragrance or flavor composition or a preparation for nutrition or pleasure.

As mentioned above the inventively used compounds of formulas (III) and (IV) are not yet described in the state of the art. A further aspect of the present invention accordingly relates to a compound of formula (III) or (IV)

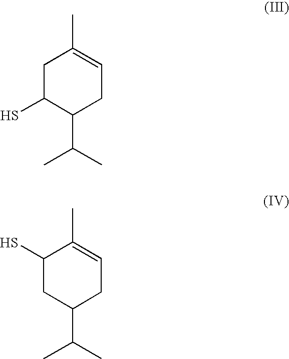

or a mixture comprising or consisting of one or more compounds selected from the group consisting of compounds of formulas (III) and (IV).

Particular preference is for an inventive mixture (as described above), further comprising one or more compounds selected from the group consisting of compounds of formulas (I) and (II)

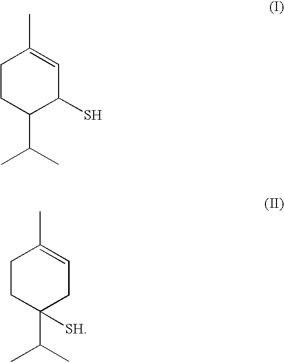

For the particularly preferred compounds of formulas (III) and (IV) or the particularly preferred mixtures and the compounds of formulas (I), (II), (III) and (IV) contained in these, that stated further above applies by analogy.

The inventively used or inventive compounds of formula (A) can advantageously be used in combination with further flavors and/or fragrances to produce flavor or fragrance compositions. To this end not only individual compounds of formula (A), but also a mixture of a plurality of compounds of formula (A), in particular a mixture as described above, can be used in combination with (further) flavors (e.g. menthene thiols), which are not compounds of formula (A). In addition, the inventive or inventively used compounds of formula (A) or inventive fragrance or flavor compositions (as described below) can be incorporated into items that are flavored or to be flavored, in particular in preparations for nutrition, oral hygiene or pleasure, or used to fragrance certain items.

In the following, inventive fragrance or flavor compositions, preparations for inventive use in nutrition, pleasure or oral hygiene and inventively perfumed items are described.

A further aspect of the present invention accordingly relates to a fragrance or flavor composition, consisting of or comprising
(i) a compound of formula (A) or a mixture of two or more compounds of formula (A), as defined further above, and
(ii) one, two, three or more further fragrances and/or flavors (preferably in a sensorially effective quantity), wherein the further fragrance(s) or flavor(s) is (are) not a compound(s) of formula (A),
   provided that the fragrance or flavor composition comprises neither p-menthene thiol-1,8 nor 1,8-epi-thio-p-methane and also provided that the fragrance or flavor composition, in the presence of hexane and/or ethyl acetate, comprises one, two or three or more further fragrances and/or flavors, wherein the further fragrance(s) and/or flavor(s) is (are) not a compound(s) of formula (A). As described further above, the present invention does not relate to a fragrance or flavor mixture that corresponds to a mixture as described in the Flavor and Fragrance Journal 1993, 8, 289-294.

Inventive fragrance or flavor compositions (as described above) preferably comprise in particular one or more (further) sensorially effective substances such as, for example, synthetic, natural or nature-identical flavors or plant extracts. The ratio of quantities between the inventive or inventively used compounds of formula (A) and the further components of an inventive fragrance or flavor composition is naturally highly dependent upon the desired sensorial overall impression of the flavor composition and can be correspondingly adjusted by a person skilled in the art according to the desired overall impression.

Particular preference according to the invention is for a fragrance or flavor composition (as described above), wherein the total quantity of compounds of formula (A) is sufficient in order, in the fragrance or flavor composition, to modify and/or intensify the sensory impression of the further or one, a plurality of or all the further fragrances and/or flavors.

Particular preference is also for an inventive fragrance or flavor composition (as described above), wherein
the further or one, a plurality of or all the further fragrance(s) is or are selected from the group consisting of flowery and fruity fragrances
   and/or
the further or one, a plurality of or all the further fragrance(s) is or are selected from the group consisting of flowery and fruity flavors.

A further aspect of the present invention relates to the use of an inventive fragrance or flavor composition (as described above) to impart, modify and/or intensify a flowery and/or fruity smell or taste note, in particular to impart, modify and/or intensify one, two or all the smell or taste notes grapefruit, tropical fruits and blackcurrant. Accordingly, particular preference is for inventive fragrance or flavor compositions (as described above), containing a quantity of inventively used or inventive compounds of formula (A), that is sufficient to impart, modify and/or intensify one or more of the smell and/or taste notes grapefruit, tropical fruits and blackcurrant, preferably all the said smell and/or taste notes.

Inventive methods for imparting, modifying and/or intensifying a smell and/or taste are described below.

For the compounds of formula (A) or mixtures preferably contained in an inventive fragrance or flavor composition (A), that stated further above applies by analogy.

The present invention also relates to a preparation for nutrition, pleasure or oral hygiene, comprising a sensorially effective quantity of
a compound of formula (A)
   or
a mixture of two or more compounds of formula (A),
   as defined above,
   or
an inventive compound of formula (III) or (IV)
or an inventive mixture
as described above,
   or
an inventive fragrance or flavor composition as described above
   and
one or more further components suitable for consumption.

For the preferably contained compounds of formula (A) or the preferably contained inventive mixtures or fragrance or flavor compositions, that stated further above applies by analogy.

The inventive preparations advantageously have particularly surprising additional and well-balanced taste impressions as well as a natural, more complex and more authentic taste profile.

The inventive fragrance and flavor compositions (as described above) also surprisingly have particularly advantageous additive and well-balanced taste impressions, gain more body and develop a natural, complex and authentic taste profile.

It is also particularly advantageous that the inventive or inventively used compounds of formula (A) are extraordinarily intensive, adherent and complex. They are therefore very economical, can be used in very low doses (to some extent in the ppb range and below) in the preparations and demonstrate a long-lasting effect.

It has also been found that the inventive or inventively used compounds of formula (A) are not only able to add to a fragrance or flavor composition (in particular one of those described above) valuable and special taste or smell notes.

In particular for inventive flavor compositions, during our own investigations, a taste-enhancing (or boosting) effect from the inventively used compound(s) of formula (A) was observed, which is characterized in particular by an overall stronger and preferably more rounded taste impression.

The inventive or inventively used compounds of formula (A) are preferably particularly well-suited to incorporation in flavor or fragrance compositions and preparations for nutrition, pleasure or oral hygiene, in which fruity and/or exotically fruity notes are desired or (should) even dominate. These include in particular the tastes strawberry, raspberry, mulberry, elderberry, gooseberry, red- and blackcurrant, bilberry, blueberry, cranberry, cherry, pear, apple, rhubarb, grape, plum, mirabelle, peach, apricot, nectarine, quince, kiwi, banana, lemon, orange, Citrus sinensis, mandarin, clementine, grapefruit, Citrus maxima, lime, sweet lime, kumquat, tangor, tangelo, star fruit, lychee, pineapple, guava, papaya, passion fruit and mango.

In an inventive flavor composition (as described above) the further or the one, more or (preferably) all the further flavors, with particular preference for two, three, four, five, six, seven, eight, nine or ten further flavors, is or are selected from the group consisting of:

acetophenone, allyl caproate, alpha-ionone, beta-ionone, anisaldehyde, anisyl acetate, anisyl formate, benzaldehyde, benzothiazole, benzyl acetate, benzyl alcohol, benzyl benzoate, beta-ionone, butyric acid, butyl butyrate, butyl caproate, butylidene phthalide, carvone, camphene, caryophyllene, cineol, cinnamyl acetate, citral, citronellol, citronellal, citronellyl acetate, cyclohexyl acetate, cymol, damascone, decalactone, acetic acid, dihydrocoumarin, dimethyl anthranilate, dimethyl anthranilate, dodecalactone, ethoxyethyl acetate, ethylbutyric acid, ethyl butyrate, ethyl caprinate, ethyl caproate, ethyl crotonate, ethyl furaneol, ethyl guajacol, ethyl isobutyrate, ethyl isovalerate, ethyl lactate, ethyl methyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyl dihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxy benzyl acetone, isoamyl alcohol, isoamyl isovalerate, isobutyl butyrate, isobutyraldehyde, isoeugenol methyl ether, isopropylmethylthiazole, lauric acid, levulinic acid, linalool, linalool oxide, linalyl acetate, menthol, menthofuran, methyl anthranilate, methylbutanol, methylbutyric acid, 2-methylbutyl acetate, methyl caproate, methyl cinnamate, 5-methyl furfural, 3,2,2-methyl cyclopentenolone, 6,5,2-methyl heptenone, methyl dihydrojasmonate, methyl jasmonate, 2-methyl methyl butyrate, 2-methyl-2-pentenoic acid, methylthiobutyrate, 3,1-methylthiohexanol, 3-methylthiohexyl acetate, nerol, neryl acetate, trans,trans,2,4-nonadienal, 2,4-nonadienol, 2,6-nonadienol, 2,4-nonadienol, nootkatone, delta-octalactone, gamma-octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethyl pentanone, thymol, delta-undecalactone, gamma-undecalactone, valencene, valeric acid, vanillin, acetoin, ethyl vanillin, ethyl vanillin isobutyrate (=3-ethoxy-4-isobutyryloxybenzaldehyde), 2,5-dimethyl-4-hydroxy-3(2H)-furanone and the derivatives thereof (in this case preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (in this case preferably ethyl maltol), coumarin and coumarin derivatives, gamma-lactones (in this case preferably gamma-undecalactone, gamma-nonalactone, gamma-decalactone), delta-lactones (in this case preferably 4-methyl delta decalactone, massoia lactone, delta decalactone, tuberose lactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, acetic acid isoamyl ester, butyric acid ethyl ester, butyric acid-n-butyl ester, butyric acid isoamyl ester, 3-methylbutyric acid ethyl ester, n-hexanoic acid ethyl ester, n-hexanoic acid allyl ester, n-hexanoic acid-n-butyl ester, n-octanoic acid ethyl ester, ethyl-3-methyl-3-phenyl glycidate, ethyl-2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanthiol, bis(2-methyl-3-furyl)disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-di methylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guajacol, 3-hydroxy-4,5-dimethyl-2(5H)-furanone, 3-hydroxy-4-methyl-5-ethyl-2(5H)-furanone, cinnamaldehyde, cinnamyl alcohol, methyl salicylate, isopulegol and stereoisomers, enantiomers, diastereomers and possibly cis/trans-isomers thereof.

Reaction mixtures, as obtainable according to the Fragrance Journal 1993, 8, 289-294 through conversion starting with respectively limonene, alpha-pinene, alpha-terpinene, gamma-terpinene or 3-carene with hydrogen sulfide in the presence of aluminum trichloride, and other mixtures disclosed there are not flavor compositions in the sense of the present invention and to that extent are excluded from the scope of protection (see above).

Because of their typical citrus and grapefruit notes the inventive or inventively used compounds of formula (A), in particular those described above as preferred, are also particularly well-suited for use in (inventive) fragrance compositions (as described above). Such fragrance compositions are preferably perfume compositions (perfume oils). Here, even in very small doses, the inventive or inventively used compounds of formula (A) allow fine, testaceous grapefruit notes to be obtained, wherein the overall taste impression is noticeably harmonized, the radiation is perceptibly increased and the adherence of the perfume compositions is considerably strengthened.

The inventive or inventively used compounds of formula (A) can be used either individually or in an optional mixture (as described in each case above) in a number of products. In a particularly advantageous way they can be combined with other fragrances in different quantity ratios to form novel perfume compositions.

Examples of fragrances with which the inventively used compounds of formula (A) can be advantageously combined and which accordingly are preferably contained in an inventive fragrance composition (as described above) can be found, for example, in S. Arctander, Perfume and Flavor Materials, Vol. I and II, Montclair, N. J., 1969, author's edition or H. Surburg, J. Panten, Common Fragrance and Flavor Materials, 5th. Ed., Wiley-VCH, Weinheim 2006.

The following in particular are mentioned:

extracts from natural raw materials such as essential oils, concretes, absolutes, resins, resinoids, balsams, tinctures such as for example
ambergris tincture; amyris oil; angelica seed oil; angelica root oil; anise oil; valerian oil; basil oil; wood moss absolute; bay oil; mugwort oil; benzoin resin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill herb oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camomile blue oil; camomile Roman oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lavage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtenol; clove leaf oil; clove flower oil; neroli oil; olibanum absolute; olibanum oil; opopanax oil; orange-flower absolute; orange oil; origanum oil; palmarosa oil; patchouli oil; perilla oil; Peruvian balsam oil; parsley leaf oil; parsley seed oil; petitgrain oil; peppermint oil; pepper oil; pimenta oil; pine oil; pennyroyal oil; rose absolute; rosewood oil; rose oil; rosemary oil; dalmation sage oil; Spanish sage oil; sandalwood oil; celery seed oil; spike lavender oil; Japanese anise oil; styrax oil; tagetes oil; fir needle oil; teatree oil; turpentine oil; thyme oil; tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom;
individual fragrances from the group of
the hydrocarbons 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenyl methane;
the aliphatic alcohols such as for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and the acetals thereof such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanaldiethylacetal; 1,1-dimethoxy-2,2,5-trimethyl-4-hexene; citronellyloxyacetaldehyde;
the aliphatic ketones and the oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds such as for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as for example 2-nonenenitrile; 2-tridecenenitrile; 2,12-tridecadienenitrile; 3,7-dimethyl-2,6-octadienenitrile; 3,7-dimethyl-6-octenenitrile;
the aliphatic carboxylic acids and the esters thereof such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate, isoamyl butyrate; hexyl butyrate; (E)- and (Z)-3-hexenyl isobutyrate; hexyl crotonate; ethyl isovalerate; ethyl-2-methylpentanoate; ethyl hexanoate; allyl hexanoate; ethyl heptanoate; allyl heptanoate; ethyl octanoate; ethyl(E,Z)-2,4-decadienoate; methyl-2-octynoate; methyl-2-nonynoate; allyl 2-isoamyl oxyacetate; methyl-3,7-dimethyl-2,6-octadienoate;
the acyclic terpene alcohols such as for example citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates, 3-methyl-2-butenoates thereof;
the acyclic terpene aldehydes and ketones such as for example geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
the cyclic terpene alcohols such as for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates, 3-methyl-2-butenoates thereof;
the cyclic terpene aldehydes and ketones such as for example menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-iron; alpha-damascone; beta-damascone; beta-damascenone; delta-damascone; gamma-damascone; 1-(2,4,4-trimethyl-2-cyclo-hexen-1-yl)-2-buten-1-one; 1,3,4,6,7,8a-hexahydro-1,1,5,5-tetramethyl-2H-2,4-a-methanona phthalen-8(5H)-one; nootkatone; dihydronootkatone; alpha-sinensal; beta-sinensal; acetylated cedar wood oil (methylcedryl ketone);
the cyclic alcohols such as for example 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as for example alpha-3,3-trimethylcyclohexyl methanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)-hexan-3-ol;
the cyclic and cycloaliphatic ethers such as for example cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropy l)-1,3-dioxane;
the cyclic and macrocyclic ketones such as for example 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 7-cyclohexadecen-1-one; 8-cyclohexadecen-1-one; 9-cycloheptadecen-1-one; cyclopentadecanone; cyclohexadecanone;
the cycloaliphatic aldehydes such as for example 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones such as for example 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert.-butyl (2,4-dimethyl-3-cyclohexen-1-yl)ketone;
the esters of cyclic alcohols such as for example 2-tert-butyl cyclohexylacetate; 4-tert-butyl cyclohexylacetate; 2-tert-pentyl cyclohexylacetate; 4-tert-pentyl cyclohexylacetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5, or 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;
the esters of cycloaliphatic carboxylic acids such as for example allyl 3-cyclohexylpropionate; allyl cyclohexyloxyacetate; cis- and trans-methyldihydrojasmonate; cis- and trans-methyljasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolan-2-acetate;
the araliphatic alcohols such as for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids such as for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers such as for example 2-phenylethyl methyl ether; 2-phenylethyl isoamyl ether; 2-phenylethyl 1-ethoxyethyl ether; phenylacetaldehydedimethylacetal; phenylacetaldehydediethylacetal; hydratropaaldehydedimethylacetal; phenylacetaldehydeglycerinacetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m-dioxine;
the aromatic and araliphatic aldehydes such as for example benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropyl phenyl)propanal; 2-methyl-3-(4-tert.-butylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as for example acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methylethyl)-1H-5-in denyl]ethanone; 5′,6′,7′,8′-tetrahydro-3′, 5′, 5′,6′,8′,8′-hexamethyl-2-acetonaphthone;
the aromatic and araliphatic carboxylic acids and their esters such as for example benzoic acid; phenylacetic acid; methyl benzoate; ethyl benzoate; hexyl benzoate; benzyl benzoate; methylphenyl acetate; ethylphenyl acetate; geranylphenyl acetate; phenylethyl-phenyl acetate; methyl cinnamate; ethyl cinnamate; benzyl cinnamate; phenylethyl cinnamate; cinnamyl cinnamate; allylphenoxy acetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as for example 2,4,6-trinitro-1,3-dimethyl-5-tert.-butylbenzene; 3,5-dinitro-2,6-dimethyl-4-tert.-butylacetophenone; cinnamic acid nitrile; 5-phenyl-3-methyl-2-pentenoic acid nitrile; 5-phenyl-3-methylpentanoic acid nitrile; methyl anthranilate; methyl-N-methyl anthranilate; Schiffs bases of methyl anthranilate with 7-hydroxy-3,7-dimethyloctanal, 2-methyl-3-(4-tert.-butylphenyl)propanal or 2,4-dimethyl-3-cyclohexenecarbaldehyde; 6-isopropylquinoline; 6-isobutylquinoline; 6-sec.-butylquinoline; indene; scatole; 2-methoxy-3-isopropylpyrazine; 2-isobutyl-3-methoxypyrazine;
the phenols, phenyl ethers and phenyl esters such as for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;
the heterocyclic compounds such as for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones such as for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,12-dodecane dioate; ethylene-1,13-tridecane dioate; coumarin; 2,3-dihydrocoumarin; octahydrocoumarin.

Reaction mixtures, as obtainable according to the Fragrance Journal 1993, 8, 289-294 through conversion starting with respectively limonene, alpha-pinene, alpha-terpinene, gamma-terpinene or 3-carene with hydrogen sulfide in the presence of aluminum trichloride, and other mixtures disclosed there are also not (inventive) perfume compositions in the sense of the present invention and to that extent are excluded from the scope of protection (see above).

In inventive perfume compositions (perfume oils) the total quantity used of compounds of formula (A) is preferably 0.00001% by weight (corresponding to 0.1 ppm) to 5% by weight, preferably 0.00005% by weight (corresponding to 0.5 ppm) to 1% by weight, more preferably 0.0001% by weight (corresponding to 1 ppm) to 0.2% by weight (corresponding to 2,000 ppm), with greatest preference for 0.0001% by weight (corresponding to 1 ppm) to 0.02% by weight (corresponding to 200 ppm), in each case in relation to the total weight of the perfume composition.

In a preferred embodiment of an inventive fragrance or flavor composition, inventive or inventively used compounds of formula (A) are preferably combined with one or more, particularly preferably with two, three, four, five, six or more, preferably flowery and/or fruity (further) fragrances.

In the course of this, through the inventively obtained compounds of formula (A) advantageously (at least to some extent) an olfactory intensification of the flowery fragrances or flavors is achieved.

Flowery fragrances or flavors, with which the inventively used compounds of formula (A) (in particular in inventive fragrance or flavor compositions) can be advantageously combined, are preferably selected from the group consisting of:

hydroxycitronellal, methoxycitronellal, cyclamen aldehyde[2-methyl-3-(4-isopropylphenyl)propanal], 1-(4-isopropyl-cyclohexyl)ethanol (Mugetanol®), 4-tert.-butyl-α-methyl dihydrocinnamaldehyde (Lilial®), cis-hexahydrocuminyl alcohol (Mayol®), 3-[4-(1,1-dimethylethyl)phenyl]propanal (Bourgeonal®), 2,2-dimethyl-3-(3-methylphenyl)propanol (Majantol®), 3-methyl-3-(3-methylbenzyl)-butan-2-ol, 2-isobutyl-4-methyltetrahydro-2H-pyran-4-ol (Florosa®), 2-methyl-3-(3,4-methylenedioxyphenyl)propanal (Heliofolal®), 4-(4-hydroxy-4-methylpentyl)-3-cyclohexene carbaldehyde (Lyral®), 4-(octahydro-4,7-methano-5H-inden-5-ylidene-butanal (Dupical®), vernaldehyde, 4-(4-methyl-3-penten-1-yl)-3-cyclohexene carbaldehyde (Vertomugal®), octahydro-5-(4-methoxybutylidene)-4,7-methano-1H-indene (Mugoflor®), 2,6-dimethyl-2-heptanol (Freesiol®), 1-ethyl-1-methyl-3-phenylpropanol (Phemec®), 2,2-dimethyl-3-phenyl-1-propanol (muguet alcohol), profarnesol, dihydrofarnesol, farnesol, nerolidol, hydroxycitronellal dimethyl acetal, hexyl benzoate, geraniol, nerol, linaool, tetrahydrogeraniol, tetrahydrolinalool, ethyl linalool, geranyl tiglinate, phenethylalcohol (2-phenylethylalcohol), citronellol, rose oxide, 2-methyl-5-phenylpentanol (Rosaphen), 3-methyl-5-phenylpentanol (Phenoxanol), methyldihydrojasmonate (Hedion®, Hedione® high cis), 2-heptylcyclopentanone (Projasmon P), cis-jasmone, dihydrojasmone, cinammic alcohol (3-phenyl-2-propen-1-ol), dihydrocinammic alcohol (3-phenylpropanol), 2-methyl-4-phenyl-1,3-dioxolan (Jacinthaflor®) and dihydromyrcenol (2,6-dimethyl-7-octen-2-ol).

Furthermore, inventive or inventively used compounds of formula (A) are advantageously suitable for intensifying the smell of fruity fragrances or flavors, in particular fruity fragrances.

Fruity fragrances or flavors, with which the inventive or inventively used compounds of formula (A) can be advantageously combined, and which accordingly are particularly preferred (further) fragrances or flavors of an inventive fragrance or flavor composition, are preferably selected from the group consisting of:

2-methyl-butyric acid ethyl ester, 4-(p-hydroxyphenyl)-2-butanone, ethyl-3-methyl-3-phenylglycidate, butyric acid isoamyl ester, acetic acid isoamyl ester, acetic acid-n-butyl ester, butyric acid ethyl ester, 3-methyl-butyric acid ethyl ester, n-hexanoic acid ethyl ester, n-hexanoic acid allyl ester, ethyl-2-trans-4-cis-decadienoate, 1,1-dimethoxy-2,2,5-trimethyl-4-hexane, 2,6-dimethyl-5-hepten-1-al, gamma-undecalactone, gamma-nonalactone, hexanal, 3Z-hexenal, n-decanal, n-dodecanal, citral, vanillin, ethyl vanillin, maltol, ethylmaltol and mixtures of these.

Inventive perfume oils (as described above) can be used in liquid form, undiluted or diluted with a solvent for perfuming (of items). Suitable solvents for this are for example ethanol, isopropanol, diethylene glycol monoethyl ether, glycerin, propylene glycol, 1,2-butylene glycol, dipropylene glycol, diethyl phthalate, triethylcitrate and isopropyl myristate.

The inventive perfume oils (as described above) can further be adsorbed by a carrier which ensures both a fine distribution of the fragrances in the product and also a controlled release of these during application. Such carriers may be porous inorganic materials such as lightweight sulfate, silica gels, zeolites, gypsums, clays, clay granules and gas concrete or organic materials such as woods and cellulose-based substances.

Inventive perfume oils (as described above) may also be in microencapsulated, spray-dried, inclusion complex or extrusion product form and in such form may be preferably added to a product to be perfumed.

If necessary the properties of perfume oils modified in this way may be further optimized through so-called “coating” with suitable materials for a controlled release of perfume, to which end preferably wax-like synthetic materials such as for example polyvinyl alcohol are used.

Microencapsulation of inventive perfume oils can for example take place by the so-called coacervation method with the help of capsule materials, for example made from polyurethane-like substances or soft gelatin. The spray-dried perfume oils can for example be produced by spray drying of a dispersion or emulsion containing the perfume oil, wherein for the carriers modified starches, proteins, dextrin and vegetable gums can be used. Inclusion complexes can for example be produced by mixing inventive perfume oils and cyclodextrins or urea derivatives in a suitable solvent, such as water. Extrusion products can result from the blending of the (inventive) perfume oils with a suitable extrudable substance and by extrusion with subsequent solidification, if necessary in a suitable solvent (e.g. isopropanol).

The inventive perfume oils (as described above) can be used advantageously in concentrated form, in solution or in modified form as described above for the production of perfumed items.

Accordingly the present invention also relates to a perfumed item comprising an inventive fragrance or flavor composition, preferably an inventive perfume composition (perfume oil). Preferred items perfumed or to be perfumed are perfume extracts, eaux de parfum, eaux de toilettes, aftershaves, eaux de colognes, pre-shave products, splash colognes and perfumed freshening wipes, acidic, alkaline and neutral cleaners, such as, for example, floor cleaners, window cleaners, dishwashing detergents, bath and sanitary cleaners, scouring milk, solid and liquid WC cleaners, pulverulent and foam carpet cleaners, liquid laundry detergents, pulverulent laundry detergents, laundry pretreatment agents, such as bleaches, soaking agents and stain removers, fabric softeners, washing soaps, washing tablets, disinfectants, surface disinfectants, and air fresheners in liquid or gel form or deposited on a solid carrier, aerosol sprays, waxes and polishes, such as furniture polishes, floor waxes, shoe creams, and bodycare compositions, such as, for example, solid and liquid soaps, shower gels, shampoos, shaving soaps, shaving foams, bath oils, cosmetic emulsions of the oil-in-water, water-in-oil and water-in-oil-in-water type, such as, for example, skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, haircare products, such as, for example, hairsprays, hair gels, hairsetting lotions, hair rinses, permanent and semipermanent hair colorants, hair-shaping compositions, such as cold waves and hair-smoothing compositions, hair tonics, hair creams and lotions, deodorants and antiperspirants, such as, for example, underarm sprays, roll-ons, deodorant sticks, deodorant creams, and products in decorative cosmetics, such as, for example, eyeshadows, nail varnishes, make-up, lipsticks, mascara, and candles, lamp oils, joss-sticks, insecticides, repellents, propellants.

Inventive preparations for nutrition, pleasure or oral hygiene (as described above) preferably contain 0.000000001% by weight (0.00001 ppm=0.01 ppb) to 0.05% by weight (500 ppm), preferably 0.0000001% by weight (0.001 ppm=1 ppb) to 0.005% by weight (50 ppm), particularly preferably 0.0000002% by weight (0.002 ppm=2 ppb) to 0.001% by weight (10 ppm) inventive or inventively used compounds of formula (A), in relation to the total weight of the preparation.

Other common raw materials, inactive ingredients and additives for staple or luxury foods can be contained in quantities of up to 99.999999% by weight, preferably 10 to 95% by weight, in relation to the total weight of the preparation. The (inventive) preparations can further contain water in a quantity of up to 99.999999% by weight, preferably 5 to 80% by weight in relation to the total weight of the preparation.

The inventive preparations for nutrition or pleasure are preferably for example baked goods (e.g. bread, dry biscuits, cakes, other baked goods), confectionery (e.g. chocolates, fruit gums, hard and soft caramels, chewing gum, liquorice), alcoholic or non-alcoholic drinks (e.g. coffee, tea, cocoa, drinks containing cocoa, wine, drinks containing wine, beer, drinks containing beer, liqueurs, whiskies, brandies, soft drinks containing fruit, isotonic drinks, refreshment drinks, nectars, fruit and vegetable juices, fruit or vegetable juice preparations), instant drinks, meat products (e.g. ham, fresh sausage or raw sausage preparations), eggs or egg products (dried egg, egg white, egg yolk), cereal products (e.g. breakfast cereals, muesli bars), milk products (e.g. milk drinks, ice milk, yoghurt, kefir, fresh cheese, soft cheese, hard cheese, dried milk powder, whey, butter, buttermilk), fruit preparations (e.g. jams, fruit ice, fruit sauces, fruit fillings), vegetable preparations (e.g. ketchup, sauces, dried vegetables), snack articles (e.g. baked or fried potato crisps or potato pulp products, corn- or peanut-based extrudates), products based on fat and oil or emulsions thereof (e.g. mayonnaise, remoulade, dressings), ready meals and soups, spices, spice mixtures and also, especially, seasonings which are used for example in the snacks sector. The (inventive) preparations within the scope of the present invention may also be used as semi-finished products for the production of further preparations for nutrition or pleasure. The (inventive) preparations within the scope of the present invention may also be in the form of capsules, tablets (uncoated and coated tablets, e.g. enteric coatings), dragees, granules, pellets, solids mixtures, dispersions in liquid phases, in the form of emulsions, in the form of powders, in the form of solutions, in the form of pastes or in the form of other swallowable or chewable preparations as food supplements.

Inventive preparations for nutrition or pleasure are preferably alcoholic or non-alcoholic drinks which also preferably comprise one or more usual common additives such as fruit components, inorganic or organic acids permitted for food purposes, antioxidants, opacifiers, loading materials, sweeteners, aromas, colorings, thickening agents, so-called “functional” additives and preservatives.

Fruit components here primarily means fruit aromas, fruit juices, fruit purées and fruit juice concentrates. Fruit juices or fruit concentrates that may be used are those with a citrus fruit base, for example orange, lemon, grapefruit and mandarin, and other fruits, for example apple, banana, grape, apricot and pineapple. In addition fruit juices and fruit juice concentrates from soft fruit, such as blackberries, gooseberries, currants, blueberries, strawberries and raspberries may be used. Furthermore, fruit juices and fruit juice concentrates from exotic fruits, for example guava, papaya, passion fruit, mango and banana can be used.

As sweetening agents common sugars (in particular sucrose), sweeteners, sugar substitutes and sweetening compositions, as well as mixtures of these, can be used.

Suitable sweeteners are in particular acesulfame-K, aspartame, cyclamate (and Na- and Ca-salts thereof), neohesperidine dihydrochalcone, sucralose and saccharine (and Na-, K- and Ca-salts thereof). Vegetable sweeteners may also be used, for instance glycyrrhizin and thaumatin. Acesulfame-K, aspartame, cyclamate, Na-cyclamate, saccharin, Na-saccharin and sucralose are particularly preferred.

Suitable sugar substitutes are sugar alcohols, such as isomatitol (E953), lactitol (E966), maltitol, manitol (E421), sorbitol (E420), xylitol (E967) as well as mixtures thereof. Furthermore, mixtures of synthetic sweetener and dearomatized or non-dearomatized concentrated fruit preparations can be used as sweetening agents.

Suitable emulsifiers in inventive drinks are for example gum Arabic or modified starches, with preference here being for starch sodium octenyl succinate (E 1450).

Inventive alcoholic or non-alcoholic drinks can also contain terpene oils. Preferred terpene oils in the context of the present invention comprise or consist of orange, lemon and/or grapefruit oil or fractions obtainable from these, preferably limonene (in particular d-limonene) and/or orange oil terpenes.

Preferred loading materials to be used are SAIB (saccharoseacetate-isobutyrate, E 444), ester gum (E 445) and to a limited extent dammar gum and BVOs (brominated vegetable oils). These allow stable drink clouding in emulsion drinks such as for example in soft drinks or fruit juice drinks.

An inventive drink usually contains one or more food acids. Preferred food acids are citric acid, tartaric acid, lactic acid, phosphoric acid and malic acid. The pH of inventive emulsion drinks is usually in the range 3 to 5.

Inventive alcoholic or non-alcoholic drinks may also contain hydrocolloids as thickening agents. Suitable thickening agents are carboxymethylcellulose, Xanthan, carob gum, gellan, guar gum, gum Arabic, carrageenan, alginic acid, alginates, pectin and mixtures of these.

Inventive alcoholic or non-alcoholic drinks can advantageously also contain so-called “functional” additives, such as for example vitamins (A, B, C, D, E, K), minerals, herb extracts, roughage, prebiotic additives, amino acids, taurine and/or caffeine.

Inventive alcoholic or non-alcoholic drinks can be colored with food colorings and/or coloring foods (e.g. beta-carotene).

Suitable preservatives are, for example, sorbic acid, benzoic acid and the alkaline salts of these.

An inventive drink can also contain natural and synthetic antioxidants. Suitable natural antioxidants are, for example, tocopherols, L-ascorbic acid, fatty acid esters of these, such as L-ascorbyl palmitate, gallic acid esters, and flavonides. Suitable synthetic antioxidants are, for example, tert.-butyl hydroxyanisole and tert.-butyl hydroquinone.

Particularly preferred preparations for nutrition or pleasure (as described above) are also soups (instant soups such as onion, leek, asparagus, pea, carrot, tomato, chicken, beef, mushroom, fish, shrimp and crab soups, as well as tinned soups of the mentioned varieties), sauces (instant sauces of the varieties beef, pork, lamb, fish, crab, shrimp, mushroom, asparagus, onion and leek), spices (bouillon of the varieties beef, pork, chicken, lamb and vegetable, seasoning mixtures of various types or spices of table salt), snacks or snack articles (baked or fried potato chips or potato pulp products, corn- or peanut-based extrudates), products based on fat or oil (mayonnaise, remoulade and dressings) and ready meals.

Inventive preparations can in particular be or be contained in seasoning mixtures. Suitable seasoning mixtures contain for example synthetic, natural or nature identical flavors as well as carriers such as for example maltodextrin, salts such as for example cooking salt, spices, such as for example paprika and pepper, sugars or sugar substitutes or sweeteners, such as for example saccharine, and taste enhancers, such as for example monosodium glutamate and/or inosine monophosphate.

The inventive preparations (as described above) can be produced by incorporating the inventive or inventively used compounds of formula (A) as a solution in the form of a mixture with a solid or liquid carrier in the preparation for nutrition, oral hygiene or pleasure. Advantageously the inventive preparations, which are thus in the form of a solution if necessary, can be converted by spray drying into a solid preparation.

In order to produce an inventive preparation (as described above) according to a preferred embodiment the inventively used compounds of formula (A) or preferably a flavor composition (as described above) and if necessary other components of the inventive preparation can also be incorporated first (that is to say prior to incorporation into the preparation) in emulsions, in liposomes, e.g. based on phosphatidylcholine, in microspheres, in nanospheres or also in capsules made from a matrix suitable for staple and luxury foods, e.g. from starch, starch derivatives, other polysaccharides, natural fats, natural waxes or from proteins, e.g. gelatin. A further preferred embodiment is characterized in that the inventive or inventively used compounds of formula (A), in particular an inventive flavor composition (as described above), are first complexed with suitable complexing agents, for example with cyclodextrins or cyclodextrin derivatives, preferably beta-cyclodextrin, and used in this form.

As other components for the inventive preparations for nutrition or pleasure, there may be used further basic substances, auxiliary substances and additives conventional for foodstuffs or snacks, for example water, mixtures of fresh or processed, vegetable or animal basic or raw substances (e.g. raw, roast, dried, fermented, smoked and/or boiled meat, egg, bone, cartilage, fish, crustaceans and shellfish, vegetables, fruits, herbs, nuts, vegetable or fruit juices or pastes or mixtures thereof), digestible or non-digestible carbohydrates (e.g. sucrose, maltose, fructose, glucose, dextrins, amylose, amylopectin, inulin, xylans, cellulose), sugar alcohols (e.g. sorbitol, mannitol, xylitol), natural or hardened fats (e.g. tallow, lard, palm oil, coconut fat, hardened vegetable fat), fatty oils (e.g. sunflower oil, groundnut oil, maize oil, thistle oil, olive oil, walnut oil, fish oil, soybean oil, sesame oil), fatty acids or their salts (e.g. potassium stearate, potassium palmitate), proteinogenic or non-proteinogenic amino acids and related compounds (e.g. taurine, creatine, creatinine), peptides, natural or processed proteins (e.g. gelatin), enzymes (e.g. peptidases, glucosidases, lipases), nucleic acids, nucleotides (inositol phosphate), taste-modulating substances (e.g. sodium glutamate, 2-phenoxypropionic acid), emulsifiers (e.g. lecithins, diacylglycerols), stabilizers (e.g. carrageenan, alginate, locust bean flour, guar flour), preservatives (e.g. benzoic acid, sorbic acid), antioxidants (e.g. tocopherol, ascorbic acid), chelators (e.g. citric acid), organic or inorganic acidifying agents (e.g. malic acid, acetic acid, citric acid, tartaric acid, phosphoric acid), bitter substances (e.g. quinine, caffeine, limonene), sweeteners (e.g. saccharine, cyclamate, aspartame, neotame, neohesperidine dihydrochalcone), mineral salts (e.g. sodium chloride, potassium chloride, magnesium chloride, sodium phosphates), substances that prevent enzymatic browning (e.g. sulfite, ascorbic acid), essential oils, plant extracts, natural or synthetic colorings or coloring pigments (e.g. carotinoids, flavonoids, anthocyanins, chlorophyll and derivatives thereof), spices, as well as fragrances, synthetic, natural or nature identical flavors and taste-imparting substances.

According to a further aspect of the present invention the inventive preparations (as described above) are preferably used as semi-finished products for flavoring (further) preparations (for example for the production of finished products).

Tooth care products (as a basis for inventive oral hygiene preparations) generally contain an abrasive system (grinding or polishing agent), such as e.g. silicas, calcium carbonates, calcium phosphates, aluminum oxides and/or hydroxyl apatites, surface-active substances, such as e.g. sodium lauryl sulfate, sodium lauryl sarcosmate and/or cocamidopropyl betaine, humectants, such as e.g. glycerol and/or sorbitol, thickeners, such as e.g. carboxymethyl cellulose, polyethylene glycols, carrageenan and/or Laponite®, sweeteners, such as e.g. saccharine, taste correctors for unpleasant taste impressions, taste correctors for other, generally not unpleasant, taste impressions, taste-modulating substances (e.g. inositol phosphate, nucleotides such as guanosine monophosphate, adenosine monophosphate or other substances such as sodium glutamate or 2-phenoxypropionic acid), cooling agents, such as e.g. menthol, menthol derivatives (e.g. L-menthol, L-menthyl lactate, L-menthyl alkyl carbonates, menthone ketals, menthane carboxylic acid amides), 2,2,2-trialkyl acetic acid amides (e.g. 2.2.-diisopropyl propionic acid methyl amides), icilin and icilin derivatives, stabilizers and active ingredients, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetyl pyridinium chloride, aluminum lactate, potassium citrate, potassium nitrate, potassium chloride, strontium chloride, hydrogen peroxide, aromas and/or sodium bicarbonate or odor correctors.

Chewing gums, as a preferred embodiment of an inventive preparation for oral hygiene (as described above), generally comprise a chewing gum base, in other words a chewable mass that becomes plastic when chewed, sugars of various types, sugar substitutes, other sweet-tasting substances, sugar alcohols, taste correctors for unpleasant taste impressions, other taste modulators for other, generally not unpleasant taste impressions, taste-modulating substances (e.g. inositol phosphate, nucleotides such as guanosine monophosphate, adenosine monophosphate or other substances such as sodium glutamate or 2-phenoxypropionic acid), humectants, thickeners, emulsifiers, aromas and stabilizers or odor correctors.

The inventive or inventively used compounds of formula (A) can in each case by present as pure substances in the form of solutions. As solvents here preference is for water, methanol, ethanol, propylene glycol, glycerin, acetone, dichloromethane, diethyl ether, hexane, heptane, triacetin, vegetable oil or fats such as for example vegetable triglycerides (if necessary kosher), supercritical carbon dioxide or also mixtures of the abovementioned solvents. The very long shelf life, which is in any event advantageous, of the pure substances of inventive or inventively used compounds of formula (A) of 12 months can, in solution, advantageously be increased to 24 months or more.

A further aspect of the present invention relates to a method for imparting, modifying and/or intensifying a smell and/or taste (preferably a smell and/or taste with a flowery and/or fruity note), wherein a quantity
of a compound of formula (A)
   or
an inventive mixture of two or more compounds of formula (A),
   as described above,
   or
an inventive compound of formula (III) or (IV)
or an inventive mixture
as described above,
   or
an inventive fragrance or flavor composition as described above
   is brought into contact or mixed with a product.

Particularly preferred is an inventive method (as described above) for imparting, modifying and/or intensifying one, two or all the smell and/or taste notes grapefruit, tropical fruits and blackcurrant.

For the compounds of formula (A) or mixtures or fragrance or flavor compositions to be used by preference, that stated further above applies in each case by analogy.

The present invention also relates to a method for modifying and/or intensifying a smell and/or taste with a flowery and/or fruity note, wherein one or more fragrance(s) or flavor(s) with a flowery and/or fruity note is or are mixed with a quantity of compounds of formula (A) which is sufficient to sensorially intensify the smell and/or taste impression of the fragrance(s) and/or flavor(s).

In the following the present invention is further explained using examples which do not in any way restrict the invention.

### EXAMPLES

Unless otherwise stated, all the following data refer to weight.

Abbreviations used: DPG=dipropylene glycol; IPM=isopropyl myristate; TEC=triethyl citrate.

The composition referred to in the following as inventive “mixture X”, according to our own investigations, contained 57.5% by weight compound (Id), 33.9% by weight compound (Ib), 3.2% by weight compound (IIId) and 2.3% by weight compound (IIIb). Such a mixture is a particularly preferred inventive or inventively used mixture. An inventive mixture X by analogy to example 1 can be obtained, wherein the column chromatography is carried out in such a way that not only a compound of formula (I), but also a compound of formula (III) is incorporated in the product fraction.

### Example 1

### Synthesis of 1-p-menthene-3-thiol (compound of formula (I))

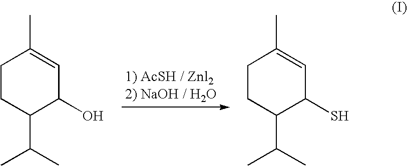

To a solution of 19.2 g (0.124 mol) piperitol in 250 ml anhydrous dichloromethane 11.7 g (0.15 mol) thioacetic acid and 20.2 g (0.06 mol) anhydrous zinc oxide were added whilst stirring. The reaction mixture obtained was stirred for 5 hours at room temperature (approximately 20° C.), and 250 ml distilled water were added, the organic phase was separated and the aqueous phase was extracted a further two times with 50 ml dichloromethane in each case.

The combined organic phases were washed with saturated aqueous sodium hydrogen carbonate solution and then saturated sodium chloride solution, dried over sodium sulfate and concentrated in the vacuum. To a stirred solution of the remaining residue (26.5 g) in 200 ml acetone 200 ml 3-molar sodium hydroxide solution was droppered in at room temperature and the reaction mixture obtained was then stirred for 4 hours at room temperature, neutralized with 3-molar aqueous hydrochloric acid and extracted three times with 50 ml dichloromethane in each case. The (subsequently combined) extracts were dried over sodium sulfate and condensed in the vacuum. Using column chromatography (silica gel, cyclohexane/ethyl acetate 3:1) from the remaining residue a specimen with compounds of formula (I) (2.72 g; purity after GC: 99.4%) was obtained.

### Example 2

### Synthesis of 1-p-menthene-4-thiol (compound of formula (II))

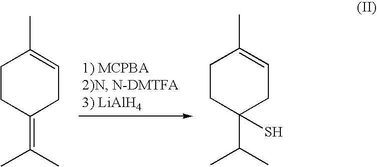

To a solution of 6.8 g (0.05 mol) terpinolene in 100 ml anhydrous dichloromethane a solution of 11.8 g (0.05 mol) m-chloroperbenzoic acid (77%) MCPBA in 150 ml anhydrous dichloromethane was droppered in whilst stirring at 0° C. and stirred for a further 30 minutes at this temperature and for a further 30 minutes at room temperature. The reaction mixture obtained was stirred for 30 minutes at room temperature with 100 ml 10% aqueous sodium carbonate solution, the organic phase was separated, washed twice with 50 ml water each time, dried over sodium sulfate and concentrated in the vacuum. The residue (12 g, containing 82% product following GC) was absorbed in 50 ml anhydrous dichloromethane and 11.6 g N,N-dimethylthioformamide (N,N-DMTFA) were added. Whilst stirring and under a nitrogen protective gas atmosphere 0.74 g trifluoroacetic acid were droppered in. The reaction mixture was then stirred for 3 hours at 60° C. under nitrogen. Following cooling to room temperature the solvent was removed in the vacuum, the remaining residue was absorbed in 200 ml n-hexane and the solution obtained washed with 300 ml water, dried over sodium sulfate and concentrated in the vacuum. A solution of 3.0 g of the residue obtained (total quantity: 8.2 g) in 5 ml tetrahydrofuran was droppered under a nitrogen protective nitrogen gas atmosphere into a boiling suspension of 0.4 g lithium aluminum hydride (LiAlH₄) in tetrahydrofuran. The reaction mixture obtained was then heated for 5 hours with reflux, following cooling to room temperature hydrolyzed with water and then concentrated in the vacuum. The remaining aqueous residue was extracted three times with 20 ml ether on each occasion. The (subsequently combined) ether extracts were washed with saturated aqueous sodium hydrogen carbonate solution, dried over sodium sulfate and concentrated in the vacuum. Through column chromatography of the residue obtained in the process (silica gel, cyclohexane/ethyl acetate 4:1) a specimen was obtained with compounds of formula (II) (1.36 g; purity after GC: 99.1%).

### Example 3

### Synthesis of 1-p-menthene-5-thiol (compound of formula (III))

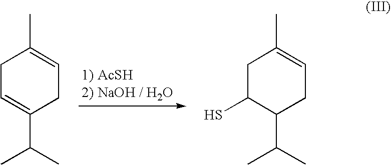

To 29.2 g (0.21 mol) gamma-terpinene 32.1 g (0.41 mol) thioacetic acid were droppered in whilst stirring at 0° C. The mixture obtained was then stirred for 5 days at room temperature, dissolved in 200 ml acetone, had 200 ml 3-molar aqueous sodium hydroxide solution added, was stirred for 5 hours at room temperature and neutralized with 3-molar aqueous hydrochloric acid. The organic phase was then separated. The aqueous phase was extracted twice with 50 ml dichloromethane in each case and the combined organic phases were dried over sodium sulfate and concentrated in the vacuum. Through column chromatography (silica gel, cyclohexane/ethyl acetate 8:1) of the residue obtained in the process a specimen was obtained with compounds of formula (III) (0.39 g; purity after GC: 99.8%).

### Example 4

### Synthesis von 1-p-menthene-6-thiol (compound of formula (IV))

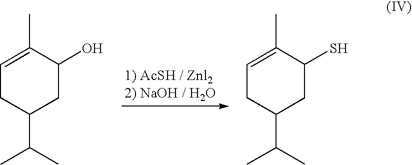

To a solution of 19.2 g (0.124 mol) 8,9-dihydrocarveol in 250 ml anhydrous dichloromethane 11.7 g (0.15 mol) thioacetic acid and 20.2 g (0.06 mol) anhydrous zinc oxide were added whilst stirring. The reaction mixture obtained was stirred for 5 hours at room temperature and then 250 ml distilled water were added. The organic phase was separated and the aqueous phase extracted a further two times with 50 ml dichloromethane in each case

The combined organic phases were washed with saturated aqueous sodium hydrogen carbonate solution and then washed with saturated sodium chloride solution, dried over sodium sulfate and concentrated in the vacuum. To a stirred solution of the remaining residue (19.7 g) in 200 ml acetone, 200 ml 3-molar aqueous sodium hydroxide solution were droppered in at room temperature. The reaction mixture obtained was then stirred for 4 hours at room temperature, neutralized with 3-molar aqueous hydrochloric acid and extracted three times with 50 ml dichloromethane in each case. The combined organic extracts were dried over sodium sulfate and concentrated in the vacuum. Through column chromatography of the residue obtained in this way (silica gel, cyclohexane/ethyl acetate 3:1) a specimen was obtained with compounds of formula (IV) (1.73 g; purity after GC: 99.6%).

### Application Examples

### Perfume Oil D: Oriental Scent for Women D/Perfume Oil P: Pink Grapefruit Scent

| | Perfume oil D | Perfume oil P |
|---|---|---|
| Component/Fragrance(s) | (inventive) | (inventive) |
| Agrunitril | | 30.0 |
| Aldehyde C8 (n-octanal) | | 4.0 |
| Aldehyde C12 (n-dodecanal) | | 2.0 |
| Aldehyde C14 so-called, 10% in TEC | 2.0 | 8.0 |
| Allyl amyl glycolate, 10% in TEC | 20.0 | 15.0 |
| Allyl capronate | | 15.0 |
| Allyl cyclohexyl propionate | | 15.0 |
| Abrin, 10% in DPG | 10.0 | |
| Benzyl acetate | | 2.0 |
| Bergamot oil | 25.0 | |
| Cashmeran ®, 10% in DPG | 6.0 | |
| Cedar wood oil | 3.0 | |
| Citral, 10% in DPG | 2.0 | |
| Citronellol | | 3.0 |
| Lemon oil, Italian | 2.0 | |
| Citrylal | | 25.0 |
| Claritone ® | | 35.0 |
| Coumarin | 12.0 | |
| Cyclamen aldehyde | 2.0 | |
| p-cymol | | 5.0 |
| Damascenone, 10% in DPG | 2.0 | |
| Beta-damascone, 10% in DPG | 5.0 | |
| Davana oil, 10% in DPG | 5.0 | |
| Gamma-decalactone, 10% in DPG | 4.0 | |
| Trans-2-decenal, 1% in DPG | | 5.0 |
| Dihydromyrceol | | 25.0 |
| Dipropylene glycol (DPG) | To 1,000 | |
| Trans-2-dodecenal, 10% in TEC | | 95.0 |
| Ebanol ®, 10% in DPG | 4.0 | |
| Ethyl butyrate, 10% in DPG | | 2.0 |
| Ethyl maltol | 6.0 | |
| Ethyl vanillin, 10% in DPG | 12.0 | 1.0 |
| Exaltenone, 10% in DPG | 2.0 | |
| Galaxolid ®, 50% in DEP | 38.0 | |
| Geranium oil | 4.0 | |
| Grapefruit base | 7.0 | 35.0 |
| Methyl dihydrojasmonate (Hedion ®) | 44.0 | |
| Cis-3-hexenol, 10% in TEC | | 2.0 |
| Cis-3-hexenyl acetate, 10% in DPG | 2.0 | |
| Cis-3-hexenyl salicylate | 2.0 | |
| Alpha-hexyl cinamaldehyde | 5.0 | |
| Iso E Super ® | 3.0 | |
| Natural spearmint oil, USA, 10% in | | 5.0 |
| TEC | | |
| Ligustral ®, 10% in DPG | 6.0 | |
| Lilial ® | 24.0 | |
| Lime oil | | 10.0 |
| Linalool | 10.0 | |
| Linalyl acetate | 12.0 | |
| Lyral | 2.0 | |
| I-menthol | | 30.0 |
| Nutmeg oil, 10% in DPG | 12.0 | |
| Nootkatone | | 1.0 |
| Brazilian orange oil | 8.0 | |
| Indonesian patchouli oil | 75.0 | |
| Resedafol | | 7.0 |
| Rose oxide, high-cis | | 4.0 |
| Sandalwood oil | 25.0 | |
| Styryl acetate | 5.0 | 3.0 |
| Terpineol | | 3.0 |
| Terpinyl acetate | 5.0 | |
| Thiocineol, 10% in TEC | | 3.0 |
| 8,3-thiomenthanone, 10% in TEC | | 5.0 |
| Thymol, 10% in TEC | | 5.0 |
| Tonalid ® | 27.0 | |
| Trans-2-tridecenal | | 1.0 |
| Triethyl citrate (TEC) | | To 1,000 |
| Vanillin | 9.0 | |
| Vetikol acetate | | 25.0 |
| Mixture X, 1% in TEC | 1.0 | 0.3 |
| TOTAL: | 1,000.0 | 1,000.0 |

In the following application examples A1 to A5, perfume oils D and P were incorporated into the corresponding formulations.

### Application Example A1

### All-Purpose Cleaner

| | | | | % by |
|---|---|---|---|---|
| Component | Manufacturer | Chemical name | Function | weight |
| De-ionized | | Water | Solvent | 59.6 |
| water | | | | |
| Mergal K9N | Troy Chemie, | 5-chloro-2- | Preservative | 0.1 |
| | Seelze | methyl-3-(2H)- | | |
| | | isothiazolone | | |
| | | and 2-methyl-3- | | |
| | | (2H)- | | |
| | | isothiazolone | | |
| Tri sodium | Various | Tri sodium | Complexing | 3.0 |
| citrate | | citrate dihydrate | agent | |
| dihydrate | | | | |
| Zetesol NL-2 | Zschimmer & | Fatty alcohol | Anionic | 30.0 |
| | Schwarz, | C12-14-sulfate, | surfactant | |
| | Germany | sodium | | |
| Imbentin | Dr. W. Kolb | Fatty alcohol | Non-ionic | 5.0 |
| C/125/055 | AG Chem. | C12-C15, 8EO | surfactant | |
| Ethanol 96% | Various | Ethanol | Solvent | 2.0 |
| Perfume oil | Symrise | | Perfume | 0.3 |
| D or P | | | (fragrance) | |

### Application Example A2

### Shampoo

| | | | % by |
|---|---|---|---|
| Component | Manufacturer | INCI name | weight |
| Deionized | | Water | 71.5 |
| water | | | |
| Plantacare | Cognis Deutschland | Sodium laureth sulfate, | 20.0 |
| PS 10 | GmbH | lauryl glucoside | |
| Euperlan PK | Cognis Deutschland | Glycol distearate, sodium | 6.0 |
| 771 | GmbH | lauryl sulfate, cocamide | |
| | | MEA, laureth-10 | |
| Dragocide | Symrise | Phenoxyethanol, | 0.5 |
| liquid | | methylparaben, | |
| | | ethylparaben, | |
| | | butylparaben, | |
| | | propylparaben, | |
| | | isobutylparaben | |
| Sodium | | Sodium chloride | 1.4 |
| chloride | | | |
| Citric acid | | Citric acid | 0.1 |
| monohydrate, | | | |
| crystalline | | | |
| Perfume oil D | Symrise | Perfume (fragrance) | 0.5 |
| or P | | | |

### Application Example A3

### Shower Gel

| | | | % by |
|---|---|---|---|
| Component | Manufacturer | INCI name | weight |
| Deionized | | Water | 76.3 |
| water | | | |
| Plantacare | Cognis | Sodium laureth sulfate, | 20.0 |
| PS 10 | Deutschland GmbH | lauryl glucoside | |
| Dragocide | Symrise | Phenoxyethanol, | 0.5 |
| liquid | | methylparaben, | |
| | | ethylparaben, | |
| | | butylparaben, | |
| | | propylparaben, | |
| | | isobutylparaben | |
| Sodium | | Sodium chloride | 1.4 |
| chloride | | | |
| Citric acid | | Citric acid | 1.3 |
| monohydrate, | | | |
| crystalline | | | |
| Perfume oil D | Symrise | Perfume (fragrance) | 0.5 |
| or P | | | |

### Application Example A4

### Fabric Conditioner

| | | | | % by |
|---|---|---|---|---|
| Component | Manufacturer | Chemical name | Function | weight |
| Deionized | | Water | Solvent | 72.4 |
| water | | | | |
| Rewoquat WE | Evonic | Dialkylesterammonium- | Cationic surfactant | 16.6 |
| 18 | Goldschmidt | ethosulfate | | |
| | GmbH | | | |
| Mergal K9N | Honeywell | 5-chloro-2-methyl- | Preservative | 0.10 |
| | Austria GmbH | 3-(2H)-isothiazolon | | |
| | | and 2-methyl-3- | | |
| | | (2H)-isothiazolon | | |
| Dow Corning | Dow Corning | Polydimethyl- | Defoaming agent | 0.30 |
| 1520 Antifoam | GmbH, | siloxane | | |
| | Germany | | | |
| Magnesium | | Magnesium | Stabilizer | 10.00 |
| chloride 1% | | chloride solution | | |
| solution | | | | |
| Perfume oil D | Symrise | | Perfume | 0.60 |
| or P | | | (fragrance) | |

### Application Example A5

### Hair Conditioner with UV Protection

| | | % by |
|---|---|---|
| Component | INCI name | weight |
| Lanette O | Cetearyl alcohol | 4.00 |
| Dragoxat 89 | Ethylhexyl isononanoate | 4.00 |
| Emulsiphos | Potassium cetyl phosphate, | 0.50 |
| | hydrogenated palm | |
| | glycerides | |
| Natrosol 250 HR | Hydroxyethyl cellulose | 0.25 |
| Neo Heliopan ® Hydro | Phenylbenzimidazole sulfonic | 2.00 |
| | acid | |
| L-arginine | Arginine | 1.20 |
| Benzophenone-4 | Benzophenone-4 | 0.50 |
| Neo Heliopan ® AP | Disodium phenyl | 0.50 |
| | dibenzimidazole | |
| | tetrasulfonate | |
| Edeta BD | Disodium EDTA | 0.05 |
| Dragocide liquid | Phenoxyethanol (and) | 0.80 |
| | methylparaben (and) | |
| | butylparaben (and) | |
| | ethylparaben (and) | |
| | propylparaben | |
| Dow Corning 949 cationic | Amodimethicone, | 2.00 |
| emulsion | cetrimonium chloride, | |
| | trideceth-12 | |
| Dow Corning 5200 | Laurylmethicone copolyol | 0.50 |
| Perfume oil D or P | Perfume | 0.95 |
| Water | Water (aqua) | To 100 |

### Application Example A6

### Chewing Gums

| | % by weight | |
|---|---|---|
| Component | A6a | A6b |
| Chewing gum base | 22.0 | 30.0 |
| Glycerin | 0.5 | 1.0 |
| Aroma of natural peppermint oil and natural spearmint | 0.7 | 0.2 |
| oil (containing l-menthol, menthyl acetate, menthone | | |
| and l-carvone) | | |
| Spray-dried l-menthol (containing 20% l-menthol) | 0.25 | |
| Glucose syrup | 16.5 | |
| Powdered sugar | To 100 | |
| Grapefruit aroma containing 1,250 ppm each of | 0.55 | — |
| compound of formula (I) and compound of | | |
| formula (III) | | |
| Aroma of passion fruit and blackcurrant with 2,400 | — | 1.10 |
| ppm compound of formula (III) | | |
| Sorbitol powder | | To 100 |
| Palatinite | | 9.5 |
| Xylitol | | 2.0 |
| Mannitol | | 3.0 |
| Aspartame | | 0.1 |
| Acesulfame K | | 0.1 |
| Emulgum/emulsifier | | 0.3 |
| Sorbitol 70%, in water | | 14.0 |

### Application Examples A7 to A11

### Flavored Preparations

A7=Instant drink powder
A8=Sugar-free instant drink powder
A9=Carbonated drink
A10=Soya fruit drink
A11=Low-fat yogurt

| | % by weight | | | | | |
|---|---|---|---|---|---|---|
| Component | A7 | A8 | A9* | | A10 | A11 |
| Compound of formula (III) | | 70 ppm | | | | |
| Compound of formula (I) | | | | | | 6 ppm |
| Mixture X | 45 ppm | | 5 | ppm | 8 ppm | |
| Sugar (saccharose) | To 100 | | 8.00 | | 5.00 | 5.00 |
| Citric acid | 4.00 | 33.33 | 0.20 | | | |
| Trisodium citrate | 0.26 | | | | | |
| Tricalcium phosphate | 0.22 | | | | | |
| Ascorbic acid (Vitamin C) | 0.24 | 0.44 | | | | |
| Opacifier and titanium | 0.20 | | | | | |
| dioxide (E 171) | | | | | | |
| Xanthan gum (E 415) | 0.072 | | | | | |
| Sodium carboxymethyl | 0.064 | | | | | |
| cellulose (E 467) | | | | | | |
| Pectin (E 440) | 0.04 | | | | | |
| Spray-dried grapefruit | 0.40 | | | | | |
| aroma with tartrazine | | | | | | |
| yellow coloring | | | | | | |
| Spray-dried blackcurrant | | 11.50 | | | | |
| aroma with red coloring | | | | | | |
| Lemon and lime aroma | | | 0.01 | | | |
| D-limonene | | | 0.005 | | | |
| Maltodextrin (powder) | | To 100 | | | | |
| Aspartame | | 3.30 | | | | |
| Hesperetin (1% in 1,2- | | | 0.05 | | | |
| propylene glycol) | | | | | | |
| Phloretin (1% in 1,2- | | | 0.05 | | | |
| propylene glycol) | | | | | | |
| Ethyl hydroxymethyl | | | 0.01 | ppb | | |
| furanone | | | | | | |
| Vanilla aroma | | | | | 0.10 | 0.125 |
| Vanillin | | | 15 | ppb | | |
| Maltol | | | 350 | ppb | | |
| 2,5-dimethyl-4-hydroxy- | | | 3 | ppb | | |
| 2H-furan-3-one | | | | | | |
| 1,2-propylene glycol | | | 0.10 | | | |
| Mixture of fruit juice | | | | | 45.00 | |
| concentrates | | | | | | |
| Soya powder | | | | | 5.00 | |
| Yogurt (1.5% fat) | | | | | | To 100 |

| | | |
|---|---|---|
| Water | To 100 | To 100 |

*Carbonization/addition of carbon dioxide following filling into bottles.

1-6. (canceled)

7. A compound of formula (III) or (IV) 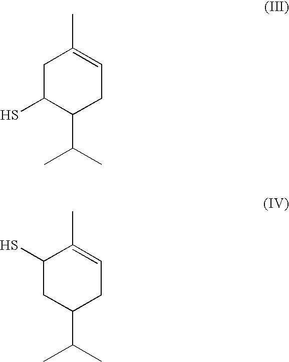
or a mixture comprising one or more compounds selected from the group consisting of compounds of formulas (III) and (IV).

8. A mixture as claimed in claim 7, further comprising one or more compounds selected from the group consisting of compounds of formulas (I) and (II)

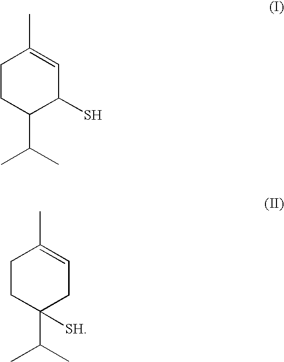

9. A fragrance or flavor composition comprising:
(i) a compound of formula (A) or a mixture of two or more compounds of formula (A) 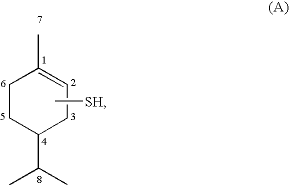
wherein the thiol group is linked with the carbon atom in position 3, 4, 5 or 6; and
(ii) one, two, three or more further fragrance(s) and/or flavor(s), wherein the further fragrance(s) and/or flavor(s) is or are not compound(s) of formula (A);
provided that the fragrance or flavor composition comprises neither p-menthene thiol-1,8 nor 1,8-epi-thio-p-methane and also provided that the fragrance or flavor composition in the presence of hexane and/or ethyl acetate comprises one, two or three or more further fragrances and/or flavors, wherein the further fragrance(s) and/or flavor(s) is (are) not a compound(s) of formula (A).

10. The fragrance or flavor composition as claimed in claim 9, wherein the total quantity of compounds of formula (A) is sufficient to modify and/or intensify the sensory impression of one, two, three or more further fragrances and/or flavors.

11. The fragrance or flavor composition as claimed in claim 10, wherein:
the one, two, three or more further fragrance(s) is or are selected from the group consisting of flowery and fruity fragrances;
and/or
the one, two, three or more further flavor(s) is or are selected from the group consisting of flowery and fruity flavors.

12. A preparation for nutrition, pleasure or oral hygiene, comprising one or more components suitable for consumption and a sensorially effective quantity of:
a compound of formula (A) or a mixture of two or more compounds of formula (A) 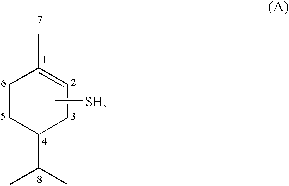
wherein the thiol group is linked with the carbon atom in position 3, 4, 5 or 6.
a compound of formula (III) or (IV) or a mixture thereof 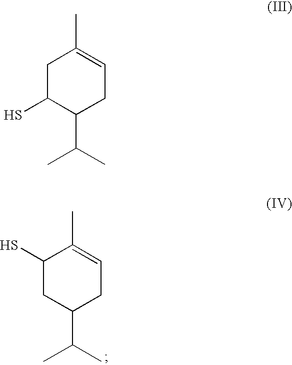
or
a mixture of compounds of formula (III) and (IV) further comprising one or more compounds formula (I) and/or (II)

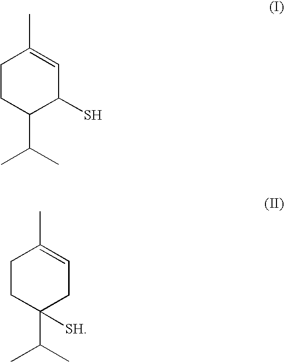

13. A perfumed item, comprising a fragrance or flavor composition as claimed in claim 9, wherein the item is selected from the group consisting of eaux de parfum, eaux de toilettes, aftershaves, eaux de colognes, pre-shave products, splash colognes, acidic, alkaline and neutral cleaners, dishwashing detergents, bath and sanitary cleaners, solid and liquid WC cleaners, liquid laundry detergents, pulverulent laundry detergents, fabric softeners, washing soaps, washing tablets, surface disinfectants, air fresheners, aerosol sprays, bodycare compositions, shower gels, shampoos, shaving soaps, shaving foams, bath oils, cosmetic emulsions, skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, hair rinses, permanent and semipermanent hair colorants, hair-shaping compositions, hair tonics, underarm sprays, roll-ons, deodorant sticks, deodorant creams, make-up, lipsticks, mascara, candles and lamp oils.

14. A method for imparting, modifying and/or intensifying a smell and/or a taste comprising bringing into contact or mixing with a product a quantity of:
a compound of formula (A) or a mixture of two or more compounds of formula (A) 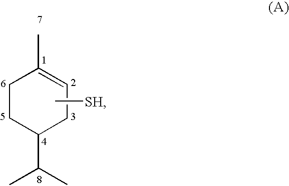
wherein the thiol group is linked with the carbon atom in position 3, 4, 5 or 6;
a compound of formula (III) or (IV) or a mixture thereof 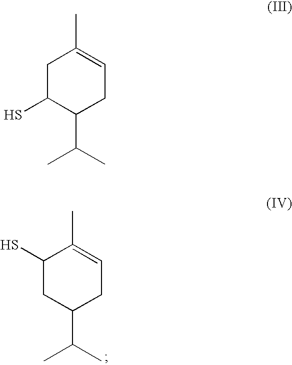
or
a mixture of compounds of formula (III) and (IV) further comprising one or more compounds formula (I) and/or (II)

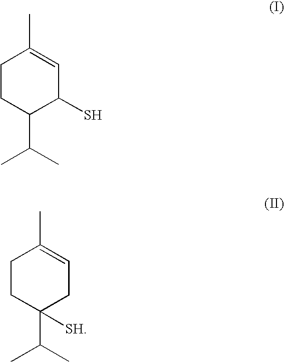

15. The method as claimed in claim 14, wherein the smell and/or taste that is imparted, modified, and/or intensified is one, two, or all of the smell and/or taste notes of grapefruit, tropical fruits, or blackcurrant.

16. The method as claimed in claim 14, wherein the compound of formula (A) or a plurality of, or all compounds of formula (A) in the mixture is/are selected independently of one another in each case from the group consisting of compounds of formulas (I) and (III)

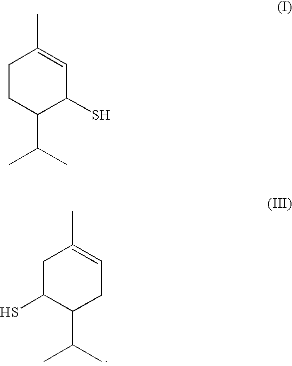

17. The method as claimed in claim 14, wherein the compound of formula (A), or a plurality of, or all compounds of formula (A) in the mixture is/are selected in each case independently of one another from the group consisting of compounds of formulas (Ia), (Ib), (Ic), (Id), (IIIa), (IIIb), (IIIc) and (IIId)

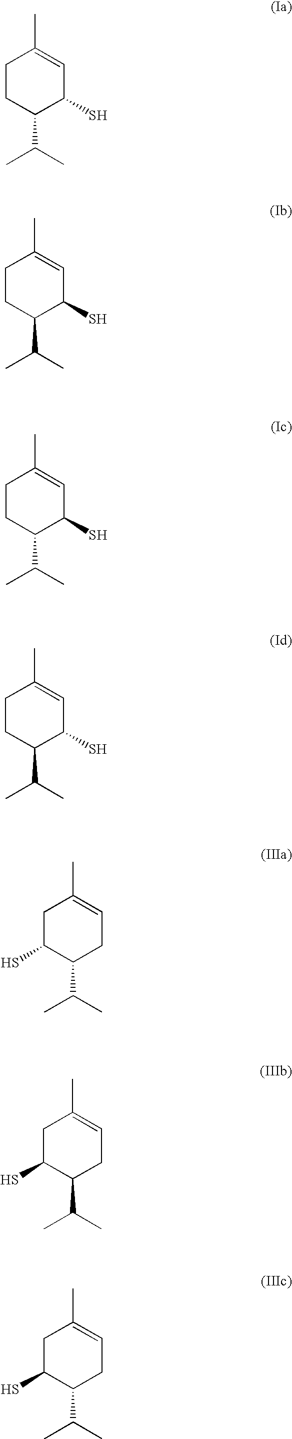

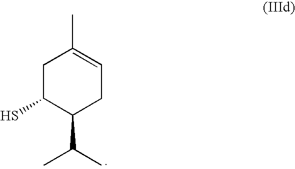

18. The method as claimed in claim 17, wherein a mixture of two or more compounds of formula (A) are brought into contact or mixed with a product, and wherein the mixture comprises:
one or more compound(s) of formula (A) selected from the group consisting of the compounds of formulas (Ia), (Ib), (Ic) and (Id); and/or
one or more compounds of formula (A) selected from the group consisting of the compounds of formulas (IIIa), (IIIb), (IIIc) and (IIId).

19. The method as claimed in claim 18, wherein the mixture comprises two or more compounds of formulas (Ib), (Id), (IIIb) and (IIId).

20. The method as claimed in claim 18, wherein the mixture comprises:
one or more compounds of formula (A) selected from the group consisting of the compounds of formulas (Ia), (Ib), (Ic) and (Id); and
one or more compounds of formula (A) selected from the group consisting of the compounds of formulas (IIIa), (IIIb), (IIIc) and (IIId); and
wherein the ratio by weight of the total quantity of compounds of formula (III) to the total quantity of compounds of formula (I) in the mixture is in the range of 1:99 to 20:80.

21. The method as claimed in claim 20, wherein the total quantity of compounds of formula (III) to the total quantity of compounds of formula (I) in the mixture is in the range of 2:98 to 10:90.

22. The method as claimed in claim 21, wherein the total quantity of compounds of formula (III) to the total quantity of compounds of formula (I) in the mixture is in the range of 3:97 to 8:92.

23. The method as claimed in claim 14, wherein the product is for nutrition or pleasure.
